(19) United States
(12) Patent Application Publication
(10) Pub. No.: US 2008/0072915 A1
(43) Pub. Date: Mar. 27, 2008

(54) Device for treating nighttime breathing problems

(76) Inventor: Jozef Frans Nelissen, Nijien (BE)

Correspondence Address: HARNESS, DICKEY & PIERCE, P.L.C., P.O. BOX 8910, RESTON, VA, 20195, US

(21) Appl. No.: 11/902,264

(22) Filed: Sep. 20, 2007

Related U.S. Application Data
(63) Continuation-in-part of application No. 10/567,372.

(30) Foreign Application Priority Data
Aug. 8, 2003 (BE) 2003/0441

(51) Int. Cl. A61F 5/56; A61C 7/08

(52) U.S. Cl. 128/848

(57) ABSTRACT

Device for treating breathing problems, comprising a shaped part fitting onto the teeth of the lower jaw, a shaped part fitting onto the teeth of the upper jaw, and adjustable connecting means between the upper and lower shaped part for forward/backward and upward/downward adjustment of the upper shaped part relative to the lower shaped part, wherein one of the shaped parts is provided at the front with a slide mechanism consisting of a slide element which is slidable laterally to a limited extent along a guide element, and the adjustable connecting means are fixedly connected to the other shaped part on one side and to the slide element on the other, thus allowing a precise forward/backward and upward/downward adjustment with a limited freedom of movement of the lower jaw in relation to the upper jaw.

### FIELD OF THE INVENTION

The invention relates to a device for treating breathing problems, in particular to an oral apparatus, comprising a shaped part fitting onto the teeth of the lower jaw, a shaped part fitting onto the teeth of the upper jaw, and adjustable connecting means between the upper and lower shaped part for forward/backward and/or upward/downward adjustment of the upper shaped part relative to the lower shaped part.

### BACKGROUND OF THE INVENTION

Night-time breathing problems, which can for instance result in snoring, sleep apnea syndrome or other sleep disorders, are a generally known problem. When a person sleeps, the rear part of the tongue may tend to slide backwards and so wholly or partially close off the pharyngeal airway. It is known to solve such breathing problems with a device which can be placed in the mouth and with which the lower jaw is placed further forward in relation to the upper jaw. The neck muscles are hereby forced into a tensioned position, whereby the tongue moves forward and the airway is left clear.

The said devices placeable in the mouth are generally known and can be found in a number of different embodiments. According to a possible embodiment, an adjustable hook is used which is connected by means of a forward/backward adjustable screw to the upper shaped part and can be coupled to the lower shaped part. Such a hook has the drawback that it still allows a certain upward/downward movement, which causes an inaccuracy in the vertical (upward/downward) adjustment. Such a hook is furthermore irritating for the tongue and can come loose during sleep.

The invention has for its object to provide a device which is more user-friendly, in particular is more agreeable to wear, and allows a precise forward/backward and/or upward/downward adjustment with a limited freedom of movement of the lower jaw in relation to the upper jaw.

### SUMMARY OF THE INVENTION

According to a first aspect of the invention, the device for treating breathing problems, comprises a first shaped part fitting onto the teeth of the lower jaw, and a second shaped part fitting onto the teeth of the upper jaw, said first and second shaped parts each having a front side located between the teeth and the lips, and a back side located behind the teeth, at the side of the inner part of the mouth. Adjustable connecting means are provided between the first and second shaped part for adjustment of the first shaped part relative to the second shaped part, said adjustable connecting means having a first end being fixedly connected to the one of said first and second shaped parts, and a second end being provided with a hook-like element. The device further comprises a guide element for said hook-like element, which guide element is provided at the front side of the other shaped part. The hook-like element and the guide-element are typically arranged for allowing coupling to each other when the hook-like element is brought substantially in front of the guide element, so that in the coupled position both the guide-element and the hook-like element are situated in front of the shaped parts, i.e. at the front side located at the side of the lips.

Such an arrangement leads to a more user-friendly device at the same time allowing an accurate adjustment of the lower jaw relative to the upper jaw. By placing the hook at the front side in the coupled position, the hook will not hinder the tongue.

According to a preferred embodiment the guide element takes the form of a horizontal rod fixed to the upper shaped part and extending parallel to the front teeth. The hook-like element preferably takes the form of an element with a horizontal elongate recess shaped for hooking over the horizontal rod.

According to a further preferred aspect of the invention the adjustable connecting means are arranged for a forward/backward and upward/downward adjustment of the first shaped part relative to the second shaped part. According to a possible embodiment the adjustable connection means comprise a horizontal adjusting screw having a first and a second outer end, for forward/backward adjustment of the one shaped part relative to the other, said screw being connected by means of a connecting piece with adjustable connection means for the upward/downward adjustment. The horizontal adjusting screw for forward/backward adjustment of the one shaped part relative to the other typically co-acts on a first outer end with a threaded passage. This threaded passage can for example be provided in the hook-like element. The horizontal adjusting screw is typically provided on its second outer end with a head, and the connecting piece is provided with a horizontally oriented hole through which the horizontal adjusting screw extends, said head preventing removal of the horizontal adjusting screw outside the hole.

Further the adjustable connecting means preferably comprise a substantially vertical adjusting screw and a threaded bore co-acting therewith for the upward/downward adjustment of the first shaped part relative to the second shaped part.

According to another aspect of the invention the device is distinguished in that one of the shaped parts is provided at the front with a slide mechanism consisting of a slide element which is slidable laterally to a limited extent along a guide element, and that the adjustable connecting means are fixedly connected to the other shaped part on one side and to the slide element on the other.

By arranging a slide mechanism between the adjustable connecting means and one of the shaped parts a limited lateral movement of the lower shaped part relative to the upper shaped part is made possible. This slide mechanism operates independently of the adjustable connecting means and thus allows a precise upward/downward and forward/backward adjustment, without this adjustment being impeded by the slide mechanism. By arranging the slide mechanism on the front of the shaped part, for instance as closely as possible to the rear side of the front teeth in the position placed in the mouth, there is more space for the tongue and the movement of the tongue is impeded as little as possible.

In the context of the patent the term Ashaped part@ must be interpreted broadly, and is understood to mean for instance a brace with a palate plate as well as a flexible bite-block.

According to a preferred embodiment the dimensions of the guide element and the slide element are closely-sized transversely of the guiding direction. The slide element is hereby substantially immobile in the plane perpendicularly of the guiding direction, and the vertical (upward/downward) adjustment is not affected by the lateral freedom of movement provided by the slide mechanism.

According to a possible variant, the guide element is a rod mounted between two points of the one shaped part, and the slide element is a hollow tube slidable around this rod. In such a variant the length of the hollow tube can be chosen as a function of the desired maximum lateral displacement. Many other variants of the slide mechanism are of course also possible which likewise fall within the scope of the invention, and some examples of which are given in the figure description.

The slide element can preferably be snapped onto the guide element. These so-called Asnap-on@ slide elements have the advantage that the shaped parts can each be placed separately in the mouth, whereafter the slide element is snapped onto the guide element.

The adjustable connecting means preferably comprise a substantially vertical adjusting screw with double screw thread for upward/downward adjustment of the upper shaped part relative to the lower shaped part, which adjusting screw co-acts on one outer end with a first threaded bore connected via a connecting piece to the slide element, and which co-acts on its other outer end with a second threaded bore provided on the front side of the other shaped part, wherein a rotation of the adjusting screw changes the distance between the lower and upper threaded bores.

According to a possible embodiment, the vertical adjusting screw is provided substantially in the middle with an encircling flange with radially directed openings in which a rod fits for the purpose of turning the adjusting screw through rotation of the rod. In this manner the vertical distance can be easily adjusted while the device is being placed in the mouth. Such adjusting screws with double screw thread are already used in orthodontics, and in particular in braces for widening the upper or lower jaw, wherein the screw is built in between two parts of a palate plate.

In order to obtain a stable vertical adjustment, the vertical adjusting screw is preferably enclosed on both sides by telescopically acting tubes.

According to a further developed embodiment of the invention, the connecting piece is provided with a horizontal adjusting screw for forward/backward adjustment of the one shaped part relative to the other, which horizontal adjusting screw co-acts on one outer end with a threaded passage mounted on the slide element. The horizontal adjusting screw is preferably provided on its other outer end with a screw head, and the connecting piece is provided with a horizontally oriented hole through which the horizontal adjusting screw extends.

In the preferred embodiment the adjustable connecting means are at least partially received in a housing which is rounded on the outside such that the device feels soft in the mouth of the patient.

### BRIEF DESCRIPTION OF THE DRAWINGS

The device will be further elucidated with reference to the annexed drawings and the associated figure description of non-limitative embodiments of the device according to the invention. In the drawing:

FIG. 1 is a perspective view of a first embodiment of a device according to the invention;

FIG. 2 is a front view of the embodiment of FIG. 1;

FIG. 3 is a rear view of the embodiment of FIG. 1;

FIG. 4 is a side view of the embodiment of FIG. 1;

FIG. 5A, B show a number of embodiment variants of the slide mechanism of the device according to the invention;

FIG. 6 is a perspective view of a second embodiment of the device according to the invention;

FIG. 7 is a perspective view of a third embodiment of the device according to the invention;

FIG. 8 is a perspective view of a fourth embodiment of the device according to the invention;

FIG. 9 is a front view of the embodiment of FIG. 8;

FIG. 10 is a rear view of the embodiment of FIG. 8.

### DETAILED DESCRIPTION OF THE PREFERRED EMBODIMENTS

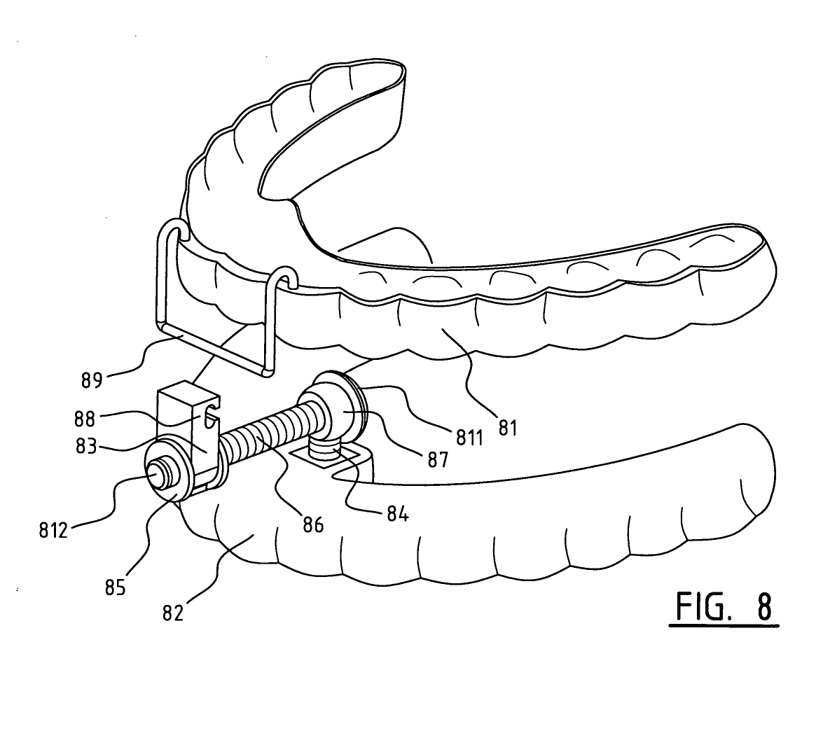

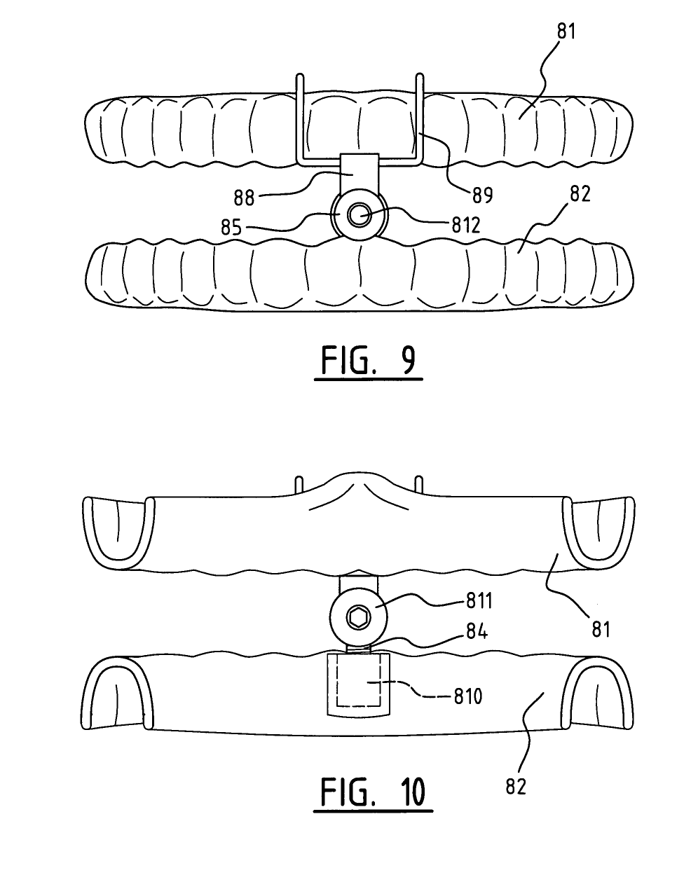

A particularly interesting embodiment of the invention is shown in FIGS. 8-10. The device shown in FIG. 8 comprises a lower shaped part 82 fitting onto the teeth of the lower jaw, and an upper shaped part 81 fitting onto the teeth of the upper jaw. The lower and upper shaped part 81, 82 each have a front side located before the teeth at the side of the lips, and a back side located behind the teeth at the side of the inner part of the mouth.

Adjustable connecting means 810, 84, 87, 86 are provided between the upper and lower shaped part for adjustment of the upper shaped part 81 relative to the lower shaped part 82. The adjustable connection means comprise a horizontal adjusting screw 86 having a first and a second outer end 811, 812. The first end 812 is connected with a hook-like coupling element 88. In the illustrated embodiment the hook-like element 88 is provided with a horizontal threaded bore for the horizontal screw 86. In that way the position of the hook-like element 88 along the horizontal screw 86 can be adjusted in function of the desired horizontal distance between the upper and the lower shaped parts 81, 82, i.e. the horizontal screw allows for forward/backward adjustment of the upper shaped part relative to the lower shaped part.

The horizontal adjusting screw is provided on its second outer end with a head 811 preventing removal of the horizontal adjusting screw outside element 87 as will be explained below.

The adjustable connecting means further comprise a substantially vertical adjusting screw 84 and a threaded bore 810 co-acting therewith for the upward/downward adjustment of the upper shaped part 81 relative to the lower shaped part 82. The threaded bore 810 is built in the lower shaped part at the back side. Rotation of the vertical screw 84 in the threaded bore 810 allows for the upward/downward adjustment.

The vertical screw is coupled to the horizontal screw by means of a ring-shaped element 87 fitting around the horizontal screw. In other words the horizontal screw is inserted through the ring-like element 87 with the head end 811 located at the back side, and the other end 812 being screwed in the hook-like element 88.

The device further comprises a guide element 89 for said hook-like element 88. The guide element is part of a bracket which is fixed in the upper shaped part 81 such that the rod-like guide element 89 extends horizontally at the front side of the upper shaped part 81. The hook-like element 88 and the guide-element 89 are arranged for allowing coupling to each other when the hook-like element is brought substantially in front of the guide element, as shown in FIG. 8. In that way both the guide-element and the hook-like element are situated in front of the shaped parts in the coupled position. This has the advantage that the tongue experiences minimal nuisance from the parts connecting the lower shaped part with the upper shaped part.

The hook-like element 88 has the form of a massive block provided at the side facing the shaped parts with a suitably shaped horizontal recess in which recess the rod-like element 89 can be fitted.

Further a protective part 85 having a smooth feel is provided around the coupling element 83, 88. This part 85 is provided with passages for easy fixation on the horizontal screw at both sides of the coupling element 83, 88.

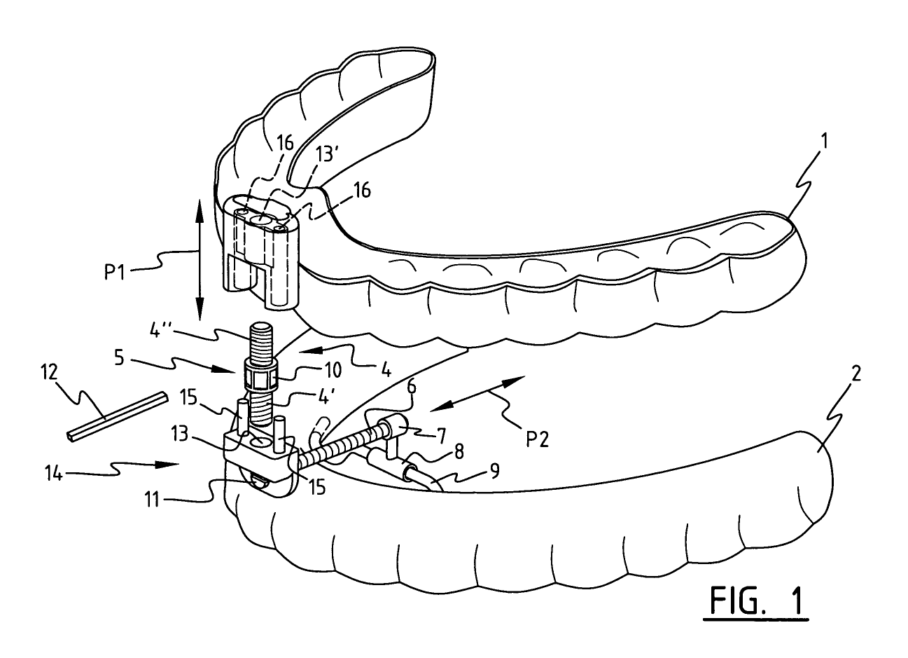

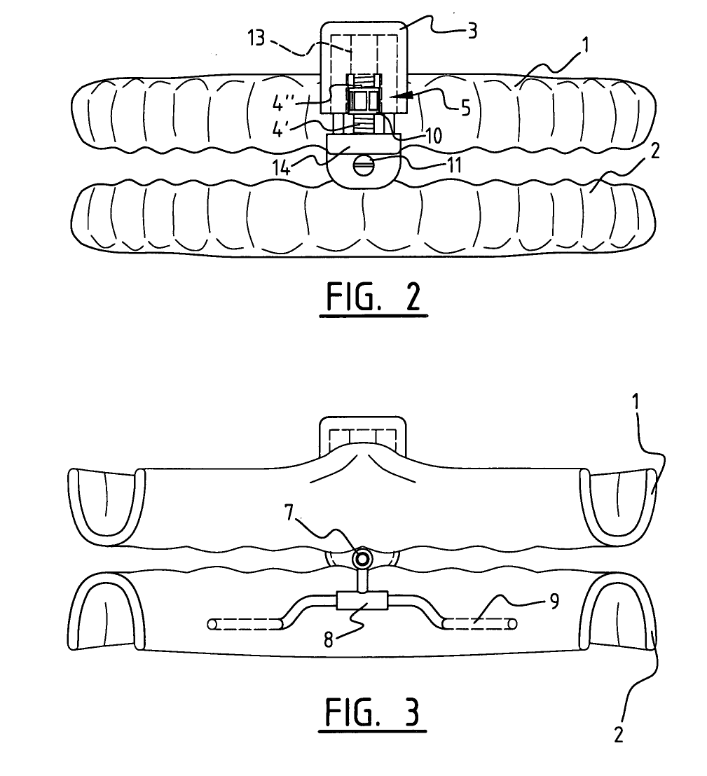

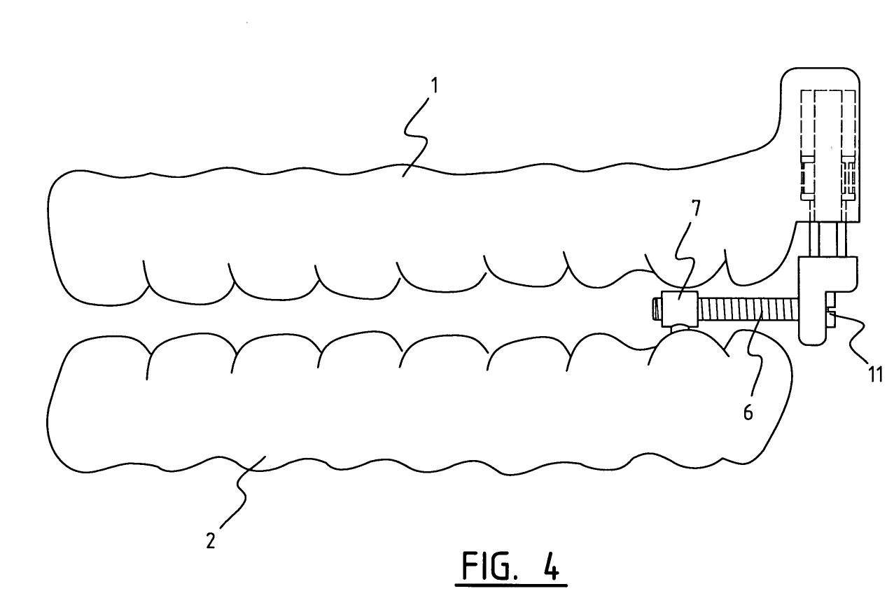

A further embodiment is shown in FIG. 1 and comprises a shaped part 2 fitting onto the teeth of the lower jaw, referred to hereafter as lower shaped part, and a shaped part 1 fitting onto the teeth of the upper jaw, referred to hereafter as upper shaped part. Note that these shaped parts can for instance also take the form of braces with a metal arch and palate plate.

The adjustable connecting means for forward/backward adjustment, as indicated with arrow P2, and for upward/downward adjustment, as indicated with arrow P1, of the upper shaped part relative to the lower will now be elucidated.

The adjustable connecting means comprise a substantially vertical adjusting screw 4 with double screw thread for upward/downward adjustment (P1). Adjusting screw 4 is provided substantially in the middle with an encircling flange 5 with radially directed openings 10 into which a rod 12 fits. This adjusting screw 4 is rotated at one end in a first threaded bore 13 and at its other end in a second threaded bore 13′ provided on the upper shaped part 1. By rotating the rod 12 the screw 4 is rotated deeper into threaded bores 13, 13′ such that the distance between the lower and upper threaded bores 13, 13′, and therefore also the distance between upper and lower shaped parts, changes. A rotation of the screw through a quarter-turn corresponds for instance to a difference in distance of 0.2 mm. In this manner the vertical distance between upper and lower shaped part can thus be adjusted precisely.

In order to impart additional strength to the vertical adjusting screw 4, it is enclosed on either side by telescopically acting tubes 15, 16. A spring can optionally be arranged in each hollow tube 16, which spring supports against the solid tube 15.

Threaded bore 13′ is received in a connecting piece 14 which is provided with a downward directed flange 17 which is substantially parallel to the front side of the shaped parts and in which a hole is arranged. Through this hole extends a horizontal adjusting screw 6 for forward/backward (P2) adjustment of the shaped parts. This horizontal adjusting screw 6 co-acts with a threaded passage 7 mounted on a slide element 8. By tightening the screw 6 it will extend further through threaded passage 7, whereby the lower shaped part 2 is pulled forward. Note that the horizontal adjusting screw 6 extends loosely through the hole in flange 17 such that it is always possible to move the lower jaw further forward than is adjusted. This movement further forward tensions the throat muscles more on the one hand, while creating a certain freedom of movement on the other, whereby the device is more joint-friendly.

The lower shaped part 2 is provided at the front with a slide mechanism which consists of a slide element 8 slidable laterally to a limited extent along a guide element 9. Guide element 9 is here a rod fixed between two points on the lower shaped part, and the slide element 8 is a hollow tube slidable around this rod. The dimensions of guide element 9 and slide element 8 are closely-sized transversely of the guiding direction, i.e. the slide element is substantially immobile in the direction perpendicular to the guiding direction. The length of hollow tube 8 can be chosen as a function of the desired maximum lateral displacement. In the case of a patient who has many problems with his/her joints it is possible for instance to opt for a relatively short tube 8 and a relatively long guide element 9 such that the lateral freedom of movement is increased.

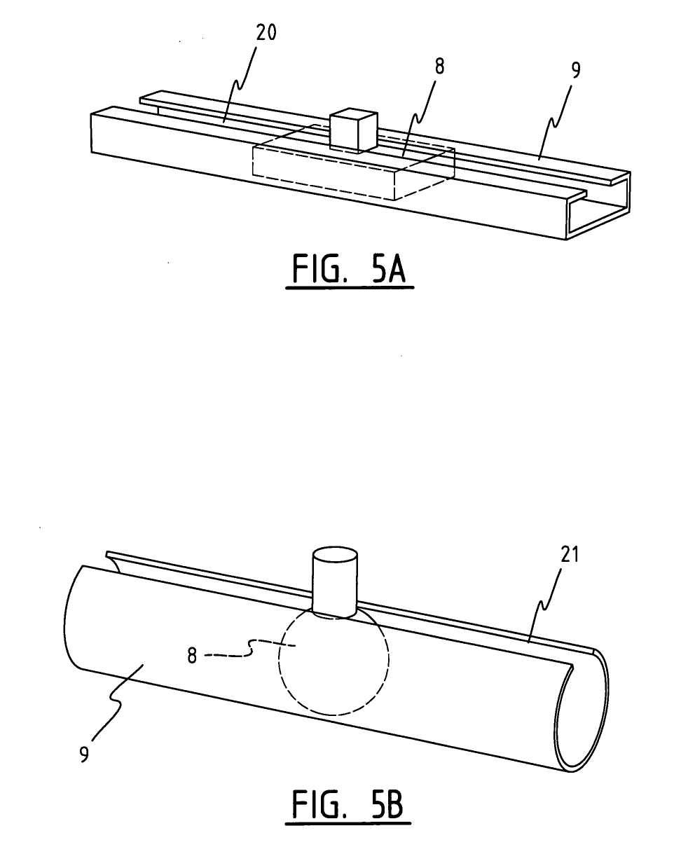

It should be understood that the slide mechanism can be embodied in many other ways; some possible embodiment variants are shown in FIGS. 5A and B.

According to the variant of FIG. 5A, a block-shaped slide element is used which is guided in a hollow tube provided on the top with a slot 20. FIG. 5B shows another variant wherein the guide element consists of a cylindrical tube provided on the top with a slot 21. Slide element 8 here takes a spherical form so that, in addition to the sliding movement, a limited swinging movement is also possible in the plane perpendicularly of slot 2 and parallel to the guiding direction.

The adjustable connecting means are fixedly connected to upper shaped part 2 on one side and to slide element 8 on the other. So as to make wearing of the device as pleasant as possible, it is recommended that the adjustable connecting means are at least partially received in a housing which is rounded on the outside.

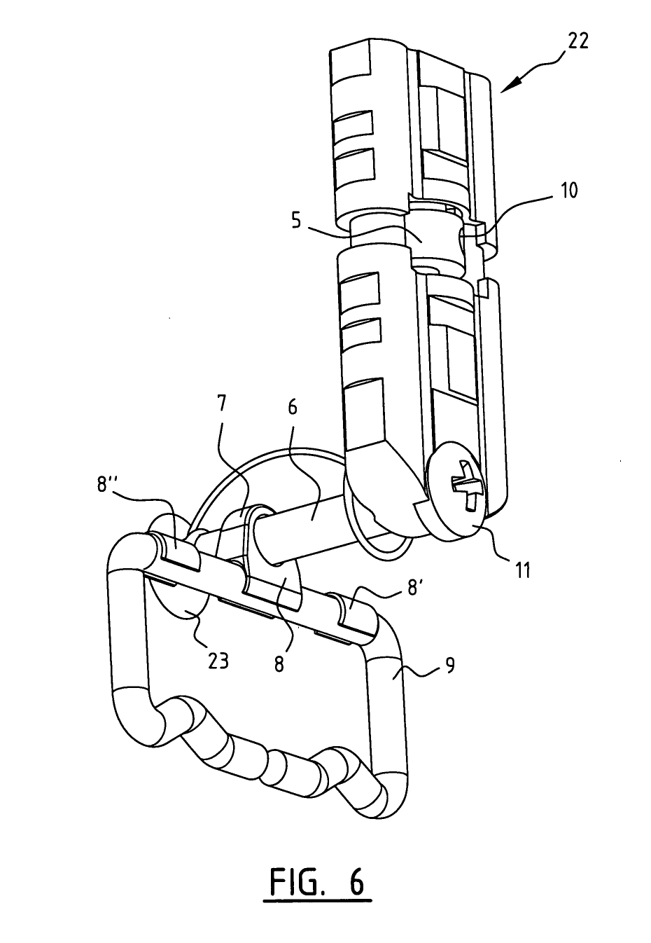

FIG. 6 shows a second embodiment variant of the device according to the invention. Guide element 9 is provided here with two extra hollow tubes 8′, 8″, whereby the lateral movement of the slide element is limited further. These hollow tubes 8, 8′, 8″ can all be embodied as snap-on elements which can be snapped easily onto the guide element.

As can be seen in FIGS. 1 and 6, the threaded bores and the vertical adjusting screw are enclosed by a housing which is rounded on the outside. In the variant of FIG. 6 the horizontal adjusting screw 6 is provided at the rear with a soft top 23, and threaded bore 7 and slide element 8 are embodied integrally without sharp edges.

The skilled person will readily appreciate that this housing can be further refined. Particularly the horizontal adjusting screw 6 with threaded bore 7 could for instance likewise be provided with a housing having a smooth feel.

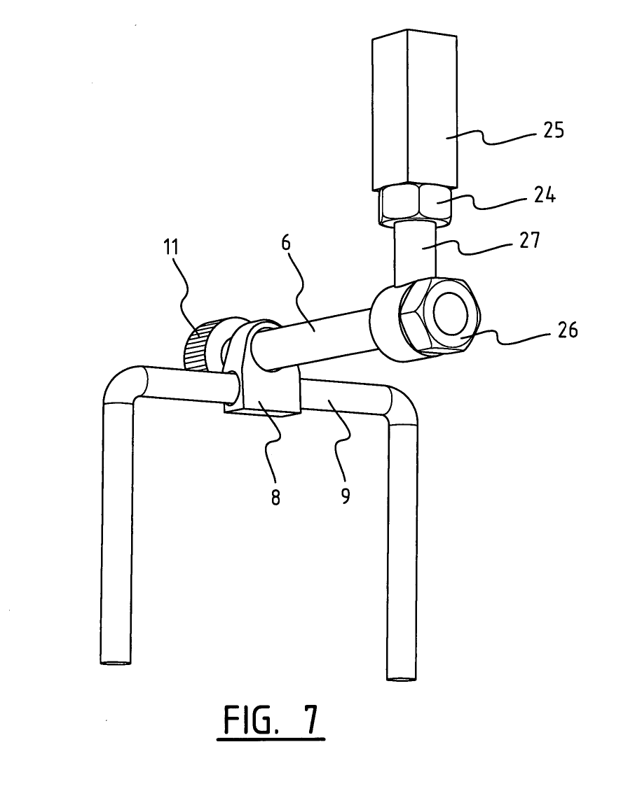

FIG. 7 shows a third embodiment variant of the device according to the invention. The slide element is here provided with a passage through which extends the guide element 9. In the third embodiment the horizontal adjusting screw 6 is placed the other way around, with its head 11 at the rear side, and a counter nut 27 is mounted on the end of the screw 6 (at the front side). To adjust the horizontal adjusting screw 6, the device has to be taken out of the mouth whereupon the position of the horizontal adjusting screw 6 can be adjusted with a screwdriver. The adjustable connecting means 22 for the upward/downward adjustment of the upper shaped part relative to the lower shaped part are even more compact in the third embodiment and are partially provided with a soft envelope. Said connecting means for the upward/downward adjustment can be partially built in the upper shaped part, so that the discomfort for the user is minimal. The adjustment connecting means comprise a vertical screw thread 27, a nut 24 fitting thereon, and a housing 25 provided with a threaded bore (not shown) co-acting with the screw thread 27. For the upward/downward adjustment the shaped parts between which the connecting means are acting, need to be taken out of the mouth and turned relative to each other around the axis of the screw thread 27, whereby the screw thread extends more or less deep in the threaded bore, i.e. in the housing. In that way the vertical distance between the upper and lower shaped part can be adjusted precisely with a very compact construction. Further there is provided a counter nut 24 which is screwed up to the housing and fixes the whole.

The scope of protection of the invention is not limited to the above illustrated embodiments, but is defined by the following claims.

1. Device for treating breathing problems, comprising:
a first shaped part fitting onto the teeth of the lower jaw, having a front side located before the teeth,
a second shaped part fitting onto the teeth of the upper jaw, having a front side located before the teeth,
adjustable connecting means between the first and second shaped part for adjustment of the first shaped part relative to the second shaped part, said adjustable connecting means having a first end being fixedly connected to the one of said first and second shaped parts, and a second end being provided with a hook-like element,
a guide element for said hook-like element, said guide element being provided at the front side of the other shaped part, wherein the hook-like element and the guide-element are arranged for allowing coupling to each other when the hook-like element is brought substantially in front of the guide element, so that in the coupled position both the guide-element and the hook-like element are situated in front of the shaped parts.

2. Device as claimed in claim 1, wherein the adjustable connecting means are arranged for a forward/backward and upward/downward adjustment of the first shaped part relative to the second shaped part.

3. Device as claimed in claim 1, wherein the adjustable connection means comprise a horizontal adjusting screw having a first and a second outer end, for forward/backward adjustment of the one shaped part relative to the other, said screw being connected by means of a connecting piece with adjustable connection means for the upward/downward adjustment.

4. Device as claimed in claim 1, wherein the adjustable connecting means comprise a substantially vertical adjusting screw and a threaded bore co-acting therewith for the upward/downward adjustment of the first shaped part relative to the second shaped part.

5. Device as claimed in claim 3, wherein the horizontal adjusting screw for forward/backward adjustment of the one shaped part relative to the other co-acts on a first outer end with a threaded passage, said threaded passage being provided in the hook-like element.

6. Device as claimed in claim 5, wherein the horizontal adjusting screw is provided on its second outer end with a head, and the connecting piece is provided with a horizontally oriented hole through which the horizontal adjusting screw extends, said head preventing removal of the horizontal adjusting screw outside the hole.

7. Device as claimed in claim 1, wherein the guide element is arranged for allowing a limited movement of the hook-like element along the guide element in a lateral direction substantially parallel to the front side of the other shaped part.

8. Device for treating breathing problems, comprising a shaped part fitting onto the teeth of the lower jaw, a shaped part fitting onto the teeth of the upper jaw, and adjustable connecting means between the upper and lower shaped part for forward/backward and upward/downward adjustment of the upper shaped part relative to the lower shaped part, wherein:
one of the shaped parts is provided at the front with a slide mechanism consisting of a hook-like element which is slidable laterally to a limited extent along a guide element,
the adjustable connecting means are fixedly connected to the other shaped part on one side and to the hook-like element on the other;
the adjustable connection means comprise a horizontal adjusting screw for forward/backward adjustment of the one shaped part relative to the other, said screw being connected by means of a connecting piece with the adjustable connection means for the upward/downward adjustment.

9. Device as claimed in claim 8, wherein the adjustable connecting means comprise a substantially vertical adjusting screw.

10. Device as claimed in claim 8, wherein the adjustable connecting means comprise a substantially vertical adjusting screw and a threaded bore co-acting therewith for the upward/downward adjustment of the upper shaped part relative to the lower shaped part.

11. Device as claimed in claim 9, wherein the vertical adjusting screw has a double screw thread for upward/downward adjustment of the upper shaped part relative to the lower shaped part, which adjusting screw co-acts on one outer end with a first threaded bore connected via a connecting piece to the slide element, and which co-acts on its other outer end with a second threaded bore provided on the front side of the other shaped part, wherein a rotation of the adjusting screw changes the distance between, the lower and upper threaded bores.

12. Device as claimed in claim 11, wherein the adjusting screw is provided substantially in the middle with an encircling flange with radially directed openings in which a rod fits for the purpose of turning the adjusting screw through rotation of the rod.

13. Device as claimed in claim 11, wherein the vertical adjusting screw is enclosed on both sides by telescopically acting tubes.

14. Device as claimed in claim 8, wherein the adjustable connection means for the upward/downward adjustment are partially built in a shaped part.
